(19) United States
(12) Patent Application Publication
(10) Pub. No.: US 2004/0116344 A1
(43) Pub. Date: Jun. 17, 2004

(54) Immunomodulatory properties of bip

(76) Inventors: Gabriel Stavros Panayi, London (GB); Valerie Mary Corrigal, London (GB); Mark Duncan Bodman-Smith, London (GB)

Correspondence Address: BANNER & WITCOFF, 1001 G STREET N W, SUITE 1100, WASHINGTON, DC, 20001, US

(21) Appl. No.: 10/471,572

(22) PCT Filed: Mar. 13, 2002
(86) PCT No.: PCT/GB02/01151

(30) Foreign Application Priority Data
Mar. 13, 2001 (GB) 0106161.3

(51) Int. Cl. A61K 38/17

(52) U.S. Cl. 514/12

(57) ABSTRACT

The present invention relates to substances having immunomodulatory properties and to the use of such substances for the treatment or prevention of an unwanted immune response. In particular, the present invention relates to the use of BiP or a functional fragment of homolog thereof, or a nucleic acid molecule encoding BiP or a functional fragment or homolog thereof, in the treatment or prevention of an unwanted immune response.

The present invention relates to substances having immunomodulatory properties and to the uses of such substances for the treatment or prevention of an unwanted immune response. The present invention also relates to a pharmaceutical composition comprising a substance having immunomodulatory properties.

There are many situations in which unwanted immune responses lead directly to disease or interfere with therapy. Autoimmune diseases are one class of diseases of the first kind. Examples of the second kind of situation are responses which interfere either with the function of transplanted tissues and organs (allo- or xeno-graft rejection) or which inactivate substances used for gene therapy and which have not previously been encountered by the immune system of the recipient. A similar example of the latter kind is the response following the infusion of therapeutic proteins collectively known as ‘biologics’ of human or non-human origin, including monoclonal antibodies and other therapeutic proteins such as blood clotting factors, and enzymes. These varied situations are at present poorly managed and each demands separate therapeutic approaches.

The therapeutic approach of the present invention is based on BiP, the 78 kD endoplasmic reticulum chaperone. In International patent application PCT/GB99/03316 (publication No. WO 00/21995), it has been demonstrated that recombinant BiP, expressed and purified from transfected E. coli was able to prevent the induction of collagen-induced arthritis (CIA) in susceptible DBA/1 mice. The isolation of BiP from human cells and cell lines has been fully described in WO 00/21995 as well as the cloning and expression of the DNA encoding this protein. The skilled person is therefore referred to WO 00/21995 for all necessary information relevant to the present application. WO 00/21955 is thus incorporated herein by reference. The BiP protein from human cells has a high degree of homology with BiP from other species and the term BiP is therefore used herein to embrace all such proteins which have the property of inducing IL-10. Minor variations on the specific DNA and amino acid sequences disclosed in WO 00/21995 are also to be included provided the above property is retained as discussed further herein.

The inventors have researched the mode of action of the BiP protein and have confirmed that it has a general immunomodulatory property which is of application to the treatment of diseases other than rheumatoid arthritis. In particular, the inventors have found that BiP can be used in preventing adjuvant arthritis (AA) in Lewis rats. As can be seen from the experiments described below, BiP was able to significantly inhibit the development of AA.

The present invention provides the use of BiP or a functional fragment or homolog thereof, or a nucleic acid molecule encoding BiP or a functional fragment or homolog thereof, in the manufacture of a medicament for the treatment or prevention of an unwanted immune response.

It is preferred that the use according to the present invention is not for the treatment or prevention of rheumatoid arthritis (RA) or collagen-induced arthritis (CIA).

It is further preferred that the use of the present invention is for preventing an unwanted immune response.

The term “BiP” as used herein refers to the 78 kD endoplasmic reticulum chaperone protein as defined in WO 00/21995. It has been found that BiP causes: CD14+ cells to release IL-10; stimulates CD8+ cells to proliferate and release IL-10; inhibits the recall antigen response; and activates the expression of an array of anti-inflammatory genes in monocytes, including the migration inhibitory factor (MIF), the soluble TNF receptor II and TIMPs. Preferably, the BiP protein has the amino acid sequence given in WO 00/21995 as SEQ ID NO. 1 or SEQ ID NO. 2.

The term “a functional fragment” as used herein refers to a fragment of BiP which is capable of eliciting at least part of an activity of the full BiP protein. In particular, it is preferred that the functional fragment has at least one of the following functions: causes CD14+ cells to release IL-10; stimulates CD8+ cells to proliferate and release IL-10; inhibits the recall antigen response; or activates the expression of an array of anti-inflammatory genes in monocytes, including the migration inhibitory factor (MIF), the soluble TNF receptor II and TIMPs. Preferably the functional fragment is at least 20 amino acids, more preferably at least 50 amino acids and most preferably at least 100 amino acids in length. Particularly preferred fragments comprise a conserved region which has been found to be homologous to a number of naturally occurring BiP proteins. Such conserved regions are considered to have a specific function.

The term “a functional homolog” as used herein refers to a homolog that retains at least part of an activity of the BiP protein described in WO 00/21995. In particular, it is preferred that the functional homolog has at least one of the following functions: causes CD14+ cells to release IL-10; stimulates CD8+ cells to proliferate and release IL-10; inhibits the recall antigen response; or activates the expression of an array of anti-inflammatory genes in monocytes, including the migration inhibitory factor (MIF), the soluble TNF receptor II and TIMPs. It is preferred that the functional homolog has at least 80%, more preferably at least 90% and most preferably at least 95% amino acid sequence homology with one of the BiP proteins described in WO 00/21995. Preferably the sequence homology is measured by using BLAST analysis. It is particularly preferred that the functional homolog differs by only 1 to 20 amino acids from one of the BiP proteins described in WO 00/21995. It is further preferred that the amino acid changes are conservative. Conservative changes are those that replace one amino acid with one from the family of amino acids which are related in their side chains. For example, it is reasonable to expect that an isolated replacement of a leucine with an isoleucine or valine, an aspartate with a glutamate, a threonine with a serine, or a similar conservative replacement of an amino acid with a structurally related amino acid will not have a major effect on the biological activity of the protein. Mutations which increase the number of amino acids which are capable of forming disulphide bonds with other amino acids in the protein are particularly preferred in order to increase the stability of the protein.

The nucleic acid molecule used in the present invention encodes BiP or a functional fragment or homolog thereof as defined above. The nucleic acid molecule can be obtained by methods well known in the art. For example, naturally occurring sequences may be obtained by genomic cloning or cDNA cloning from suitable cell lines or from DNA or cDNA derived directly from the tissues of an organism, such as a human or mouse. Positive clones may be screened using appropriate probes for the nucleotide molecule desired. PCR cloning may also be used. The probes and primers can be easily generated given that the sequence of BiP is known (see WO 00/21995). Preferably the nucleic acid molecule has the sequence given in WO 00/21995 as SEQ ID NO. 3.

Numerous standard techniques known in the field of molecular biology may be used to prepare the desired nucleic acid or the probes and primers for identifying the positive clones. The nucleotide molecules probes or primers may be synthesised completely using standard oligonucleotide synthesis methods, such as the phosphoramidite method.

Numerous techniques may be used to alter the nucleic acid sequence obtained by the synthesis or cloning procedures, and such techniques are well known to those skilled in the art. For example, site directed mutagenesis, oligonucleotide directed mutagenesis and PCR techniques may be used to alter the DNA sequence. Such techniques are well known to those skilled in the art and are described in the vast body of literature known to those skilled in the art, for example Sambrook et al., (1989).

The nucleic acid is preferably in the form of a vector comprising the necessary elements leading to the expression of the nucleic acid sequence encoding BiP or a functional fragment or homolog thereof. For example, it is preferred that the vector comprises a promoter operably linked to the nucleic acid sequence and a transcription termination sequence. Suitable promoters, transcription termination sequences and other functional elements required to obtain expression of the nucleic acid are well known to those skilled in the art.

The nucleic acid may be delivered to the individual using any method. For example, the nucleic acid may be delivered as a free nucleic acid, in the form of a viral delivery vector such as an adenovirus, contained in a liposome or via any known method.

The unwanted immune response may be any unwanted immune response. Specific unwanted immune responses are discussed in details below.

A Prevention of Unwanted Immune Responses

i) Prevention of Rejection of Allo- and Xeno-Transplants

The rejection of allo- and xeno-transplants (TX) is a major problem barrier in the more effective use of TX for the therapy of organ failure. Present anti-rejection regimens are expensive, require life-long administration, may produce toxic side effects and are not universally effective. TX are grafted at a precisely known time. Just as the inventors have shown that BiP is able to prevent CIA and AA if given, respectively, at the time of or before the induction of arthritis, it is recommended to administer BiP just before or at the time of TX to prevent rejection. TX that may be beneficially treated in this way include all TX of tissues and organs, whether solid (for example, liver, kidney) or single cell (for example, blood cells, bone marrow cells or stem cells).

ii) Prevention of Immune Response to Biologic Therapeutic Substances

A range of biologic therapies are used in clinical medicine. These include products from non-human sources and products from human sources. The biologics may be purified from a natural source, produced by recombinant gene technology, secreted after transfection of genes, or synthesised. These biologics may be proteins, glycoproteins or complex sugars. A disadvantage is that this has the potential to induce an immune response when administered to an immunologically naïve individual.

There are two main consequences of the induction of such an immune response. The first is the development of anaphylactic shock that may be life threatening. The second is the loss of therapeutic activity of the product because of the development of neutralising antibodies during the course of the immune response. It is recommended to administer BiP before or at the time of these interventions in order to prevent the development of this unwanted immune response and thus prevent these therapeutic failures.

B Treatment of Existing Diseases

Existing immune-mediated diseases such as type I diabetes mellitus (in the early phase before complete destruction of the beta cells in the islets of Langerhans), thyroiditis, multiple sclerosis and diseases in which the immune system is activated are also open to treatment by the parenteral administration of BiP. This is because BiP releases IL-10 and other regulatory molecules from target cells such as CD14+ monocytes and CD8+ T cells.

According to a first embodiment of the use of the present invention the unwanted immune response is associated with an immune-mediated disease. Immune-mediated diseases include auto-immune diseases. Specific immune-mediated diseases include type-1 diabetes, thyroididtis, multiple sclerosis, systemic lupus erythematosus, Crohn's disease and all forms of viral and autoimmune hepatitis.

It is preferred that the use according the first embodiment of the present invention additionally comprises the use of an agent for enhancing the treatment or prevention of the immune-mediated disease in the manufacture of the medicament.

The agent for enhancing the treatment or prevention of the immune-mediated disease may be any agent including IL-10, IL-4, IL-11, TGF-beta, IL-13 and soluble cytokine receptors such as IL-1Ra, IL-1 and TNF soluble receptors.

Preferably the medicament is for administration to an individual suffering from or susceptible to developing an immune-mediated disease. Methods are know for determining whether an individual is suffering from an immune-mediated disease and methods are known for determining if an individual is likely to develop an immune-mediated disease. Methods for determining whether an individual is likely to develop an immune include analysing risk factors such as genetic markers or environmental influences such as diet, etc.

In use, the medicament obtained by the use according to the first embodiment of the present invention is preferably administered to an individual before the immune-mediated disease develops or as soon as the immune-mediated disease has been diagnosed.

According to a second embodiment of the use of the present invention the unwanted immune response is associated with the rejection of a transplanted organ, tissue or cells.

The rejection response is well know and occurs when donated tissue is recognised as foreign by the recipient's immune system. The rejection response occurs with transplanted organs, such as heart, lung, kidney, liver, etc., transplanted tissues, such as skin, muscle tissue, etc., and with transplanted cells, such as bone marrow cells and stem cells.

It is preferred that the use according the second embodiment of the present invention additionally comprises the use of an agent for enhancing the treatment or prevention of the immune response associated with rejection of a transplanted organ, tissue or cells, in the manufacture of the medicament.

The agent for enhancing the treatment or prevention of the immune response associated with the rejection of transplanted organs, tissue or cells may be any agent that suppresses the immune system including glucorticoids, cyclosporin A, azathioprine, rapamycin and tacrolimus.

In use, the medicament obtained by the use according to the second embodiment of the present invention is preferably administered to an individual before or at substantially the same time as the transplantation of the organ, tissue or cells.

According to a third embodiment of the use of the present invention the unwanted immune response is the immune response to a biologic.

A biologic is any therapeutic agent given to an individual. The biologic may be from non-human or human sources. The biolgic may be a protein molecule (i.e. an enzyme, an antibody molecule, receptor ligand, etc), a glycoprotein, a polypeptide, peptide, carbohydrate, or an organic or inorganic chemical compound.

The use of biologics can cause unwanted immune responses. For example, an immune response can be raised against the biologic which may prevent the therapeutic activity of the biologic. Alternatively, the immune response may be so large that it lead to anaphylatic shock. For example, anti-TNFα therapy has resulted in the shortening of the interval between dosing (infliximab) this increasing the cost, and its use has been limited by anaphylaxis.

It is preferred that the use according the third embodiment of the present invention additionally comprises the use of an agent for enhancing the treatment or prevention of the immune response to the biologic.

The agent for enhancing the treatment or prevention of the immune response to the biologic may be any agent that suppresses the immune system including glucorticoids, cyclosporin A, azathioprine, rapamycin and tacrolimus.

In use, the medicament obtained by the use according to the third embodiment of the present invention is preferably administered to an individual before or at substantially the same time as the biologic.

The present invention also provides the use of BiP or a functional fragment or homolog thereof, or a nucleic acid molecule encoding BiP or a functional fragment or homolog thereof, for stimulating the release of IL-10 from cells capable of releasing IL-10. Preferably the cells are peripheral blood mononuclear cells (PBMCs). Preferably, the PMBCs are CD14+ monocytes and/or CD8+ T cells and/or CD4+ T cells.

Preferably BiP or a functional fragment or homolog thereof, or a nucleic acid molecule encoding BiP or a functional fragment or homolog thereof, is used to stimulate the release of IL-10 from PBMCs in vitro or ex vivo.

Preferably BiP or a functional fragment or homolog thereof, or a nucleic acid molecule encoding BiP or a functional fragment or homolog thereof is used to additionally stimulates gene expression of at least one of monocyte migration inhibitory factor (MIP), soluble TNF receptor II, IL-10 anti-inflammatory mediators and tissue inhibitor of matrix metalloproteinases (TIMP). Preferably BiP or a functional fragment or homolog thereof, or a nucleic acid molecule encoding BiP or a functional fragment or homolog thereof, does not stimulate gene expression of matrix metalloproteinases (MMPs), monocyte chemoattractant protein (MCP-1) or TNFα.

The present invention also provides a pharmaceutical preparation comprising BiP or a functional fragment or homolog thereof, or a nucleic acid molecule encoding BiP or a functional fragment or homolog thereof, in combination with a pharmaceutically acceptable carrier for use in the treatment or prevention of an unwanted immune response.

Preferably the pharmaceutical composition additionally comprises an agent for enhancing the treatment or prevention of the unwanted immune response.

The pharmaceutical composition of the present invention comprises a therapeutically effective amount of BiP or a functional fragment or homolog thereof, or a nucleic acid molecule encoding BiP or a functional fragment or homolog thereof. The term “therapeutically effective amount” as used herein refers to an amount of a therapeutic agent needed to treat or prevent the unwanted immune response.

For any agent, the therapeutically effective dose can be estimated initially either in cell culture assays, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg.

Pharmaceutical compositions of this invention comprise BiP or a functional fragment or homolog thereof, or a nucleic acid molecule encoding BiP or a functional fragment or homolog thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. Oral administration or administration by injection are preferred. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrastemal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a novel agent of this invention with a suitable non irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the novel agents of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active agent suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The present invention also provides a method of treating or preventing an unwanted immune response comprising administrating to an individual in need of such treatment an effective dose of the pharmaceutical composition according to the present invention.

As indicated above, there are many routes of administration of BiP or a functional fragment or homolog thereof, or a nucleic acid molecule encoding BiP or a functional fragment or homolog thereof, including intravenous, intramuscular, nasal, oral, cutaneous, and topical. In particular, details of several preferred approaches to using BiP or a functional fragment or homolog thereof, or a nucleic acid molecule encoding BiP or a functional fragment or homolog thereof are described below.

(a) Induction of Mucosal Tolerance.

Delivery of BiP (p78) autoantigen or functional peptides derived therefrom by mucosal routes, e.g. through the intestine or nasal mucosa, alters the immune response by downregulating disease activity leaving the patient's immune system otherwise intact. Alternatively p78 or functional p78 peptides can be delivered as a nucleic acid molecule encoding them within an appropriate mammalian expression vector.

(b) Vaccination with TCR Peptides

Peptides of the CDR3 region of the T cell receptor Valpha and Vbeta chains can be synthesised and used as vaccines for delivery by intradermal or intramuscular injection (see Kotzin et al., Arthritis Rheum., 11, 1906-1919, 1998). BiP or a functional fragment or homolog thereof can be used in the same way.

(c) MHC Blockade with Native or Altered Peptides

BiP or a functional fragment or homolog thereof, may be given parenterally or orally in appropriate cases either unmodified or modified by amino acid substitution and/or attachment of chemical groupings so as to block MHC and especially HLA-DR4 thereby leading to suppression of T cell activation and disease. BiP or a functional fragment or homolog thereof, may be combined with soluble HLA-DR4 molecules and applied parenterally or orally.

(d) Induction of Tolerance by Plasmid DNA Immunisation

Plasmids consisting of nucleic acid coding for BiP or a functional fragment or homolog thereof, may be given by injection. DNA coding for human IL-10, IL-4, IL-11, or TGF-beta, incorporated singly or in any combination, may be used to deviate the immune response to BiP towards a TH2 mode so as to suppress disease.

The present invention is now described by way of example only with reference to the following Figures:

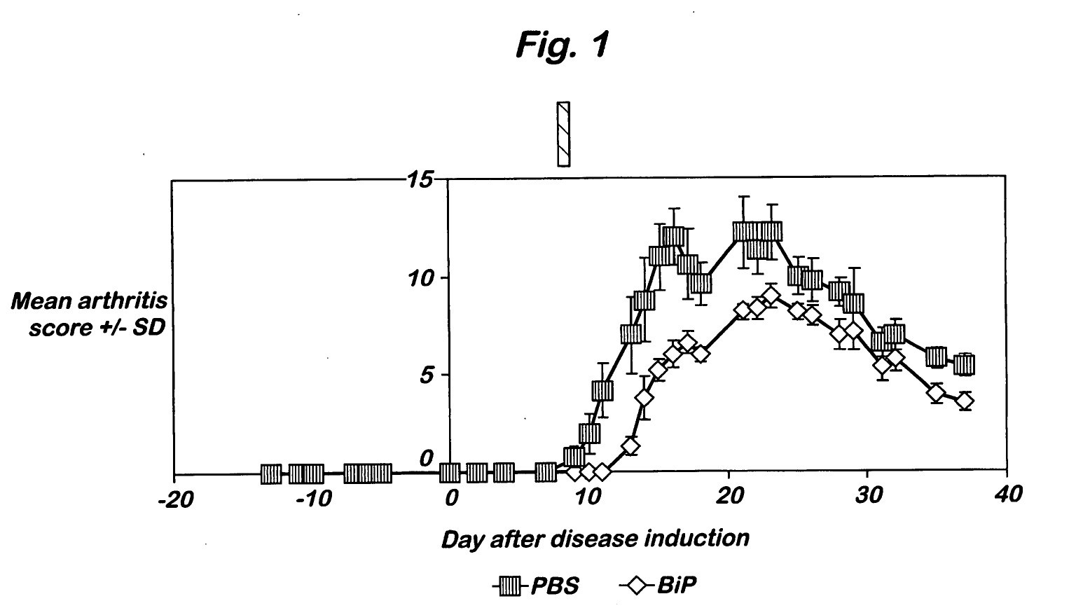

FIG. 1 shows the prevention of adjuvant arthritis by BiP. Male Lewis rats (n=5) were immunised with 50 μg PBS/DDA in each hind footpad (ie 100 μg/rat). Control animals (n=5) received only the PBS/DDA mixture. Thirteen days later, adjuvant arthritis was induced by a single intradermal injection of 0.5 mg M. tuberculosis in 100 μl IFA in the base of the tail.

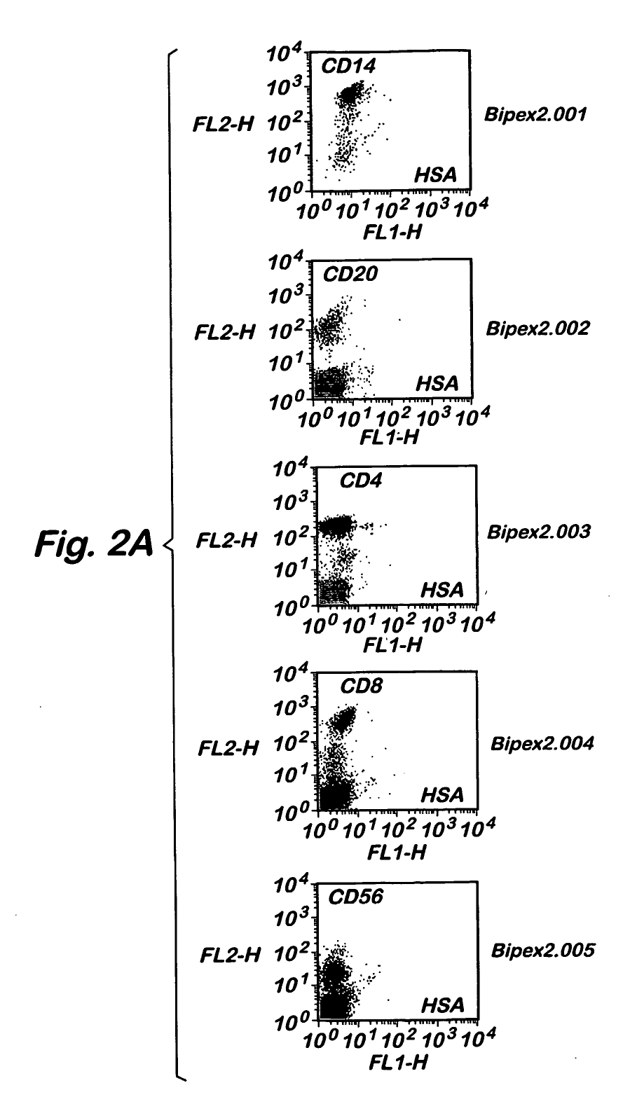

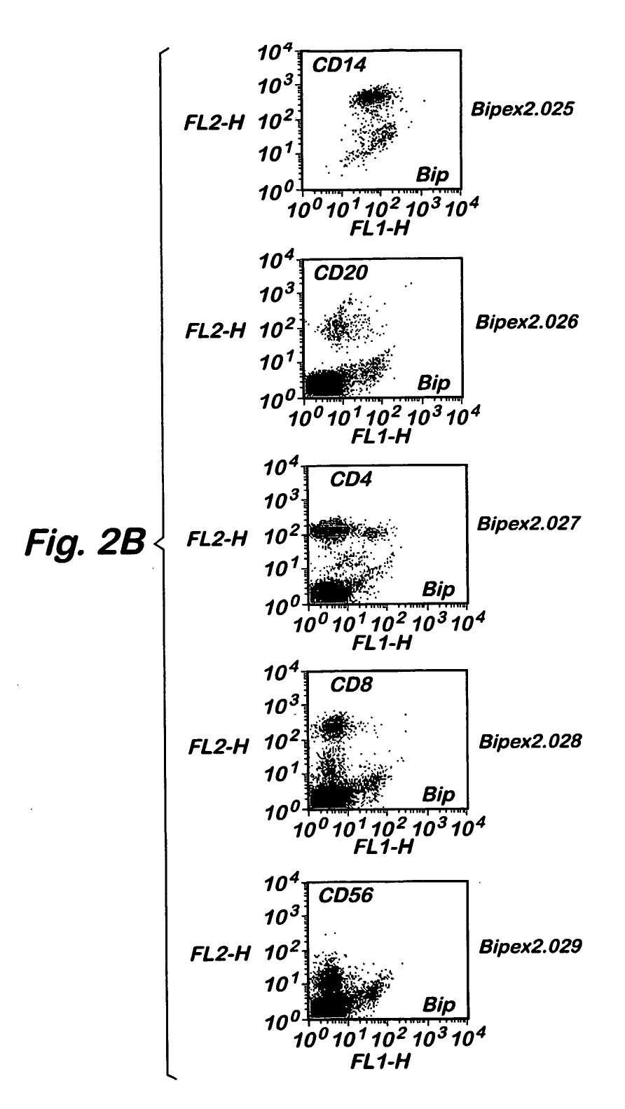

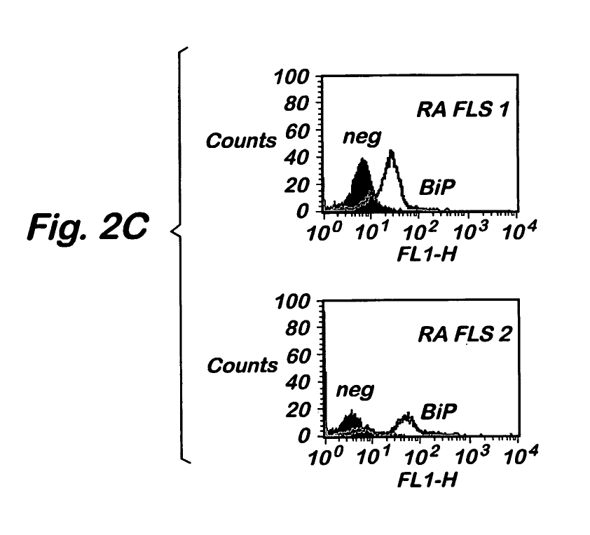

FIG. 2 shows the results of the investigation into BiP binding to peripheral blood mononuclear cell (PBMC) populations and fibroblast like synoviocytes by double immunofluorescence. Column A shows PBMC stained with human serum albumin (HSA) fluorescein isothiocyanate (FITC), as the negative control, and column B, FITC conjugated BiP, the PBMC were double stained with CD14, CD20, CD4, CD8, CD56. Column C shows BiP.FITC binding to 2 rheumatoid arthritis fibroblast-like synoviocytes compared with the HSA.FITC negative control.

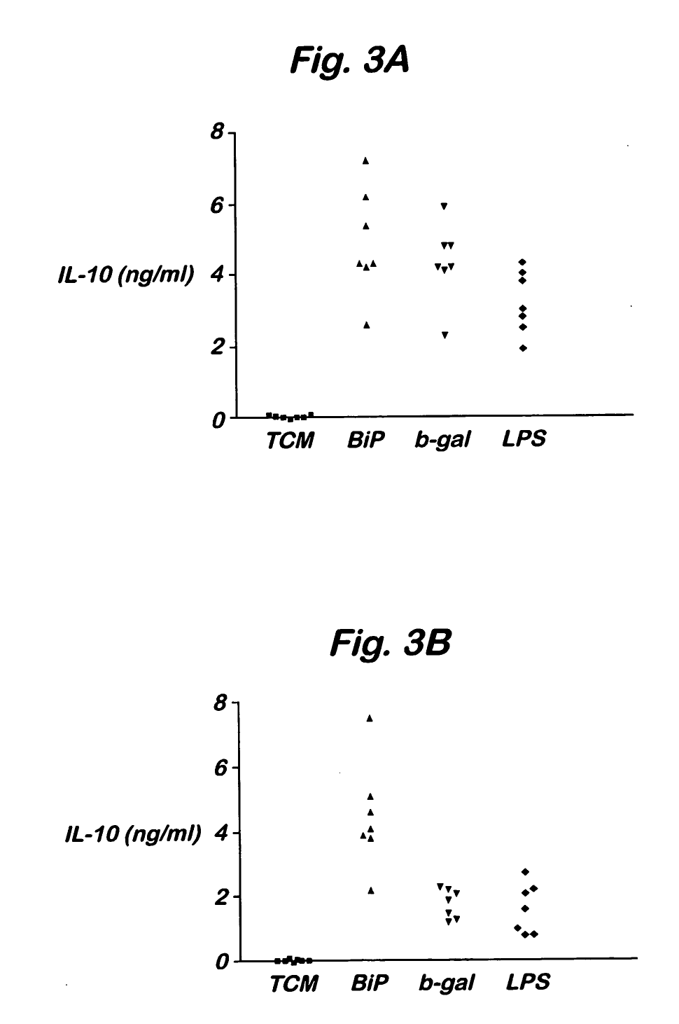

FIG. 3 shows IL-10 production following culture of peripheral blood mononuclear cells (PBMC) with BiP (20 μg/ml), beta-galactosidase (b-gal) (20 μg/ml) or lipopolysaccharide (LPS) (20 ng/ml) in the absence (A) or presence (B) of polymixin B for 24 hours. Culture supernatants were collected and IL-10 was measured by ELISA.

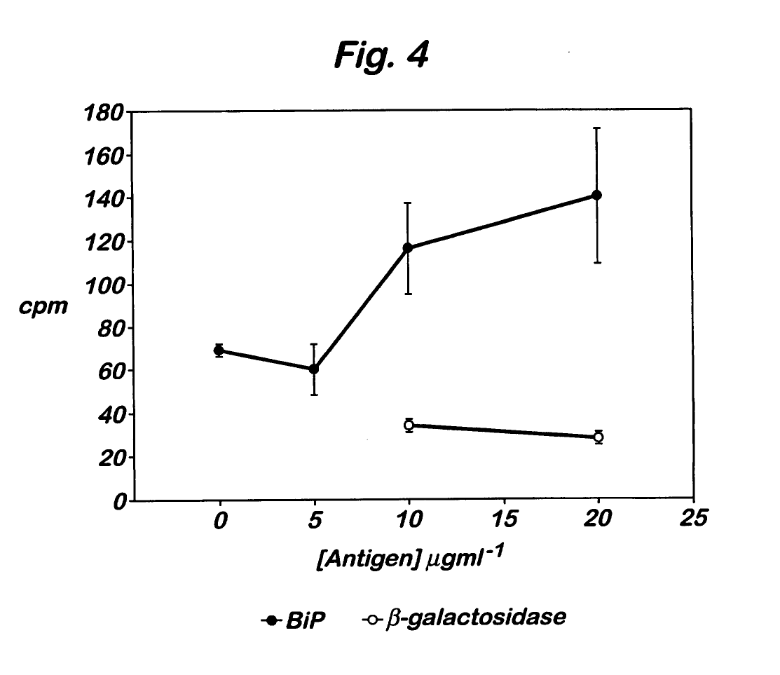

FIG. 4 shows the proliferation of CD8 clone FC2B5 to BiP (closed circles) and control antigen β-galactosidase (open circles). This clone was generated from the peripheral blood of a normal individual. This profile is representative of other BiP responsive clones.

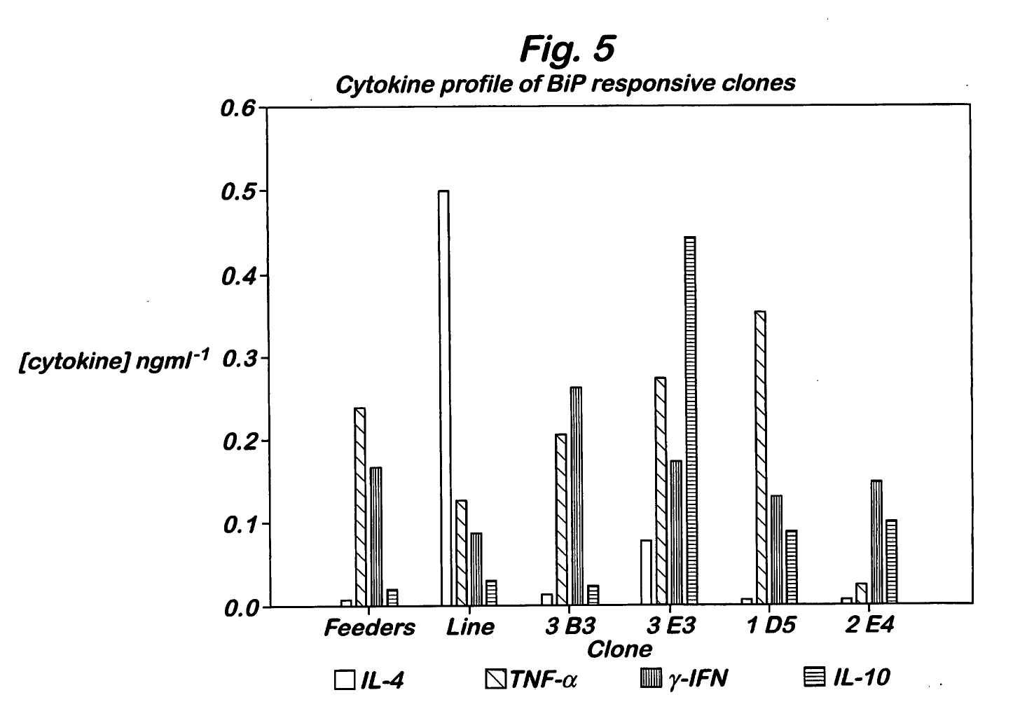

FIG. 5 shows the cytokine profiles of BiP responsive clones and lines. The cytokine levels were measured in supernatants of cells previously shown to be BiP responsive, stimulated by mitogen. The profiles are compared with irradiated feeder cells alone (first data set).

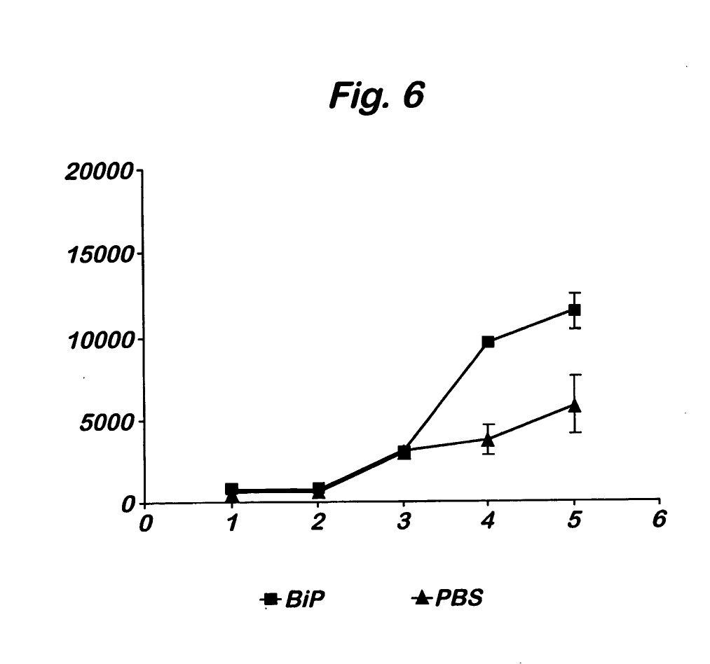

FIG. 6 shows the proliferative response of T cells to BiP stimulation.

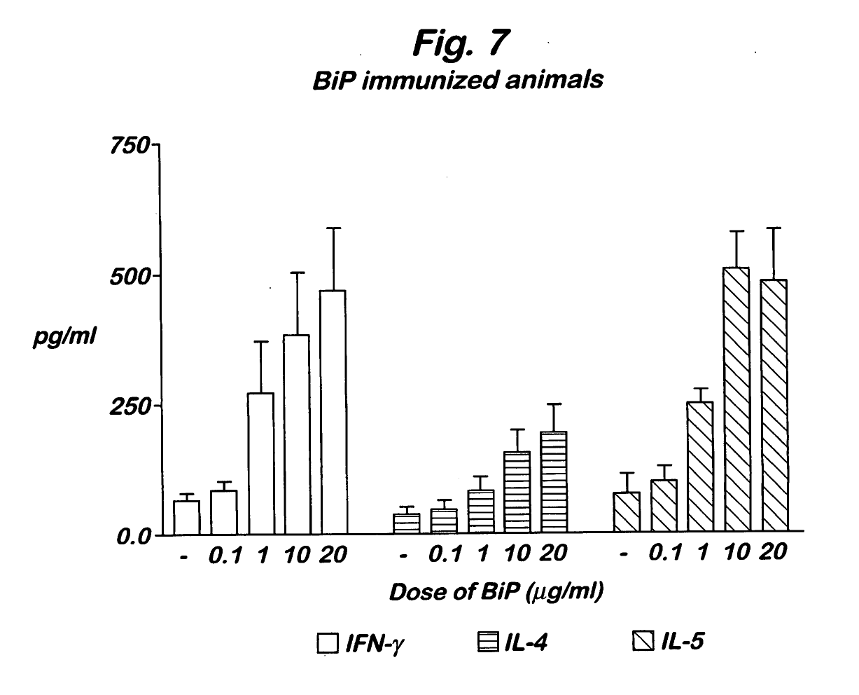

FIG. 7 shows BiP-driven T cell cytokine production from animals immunised with BiP.

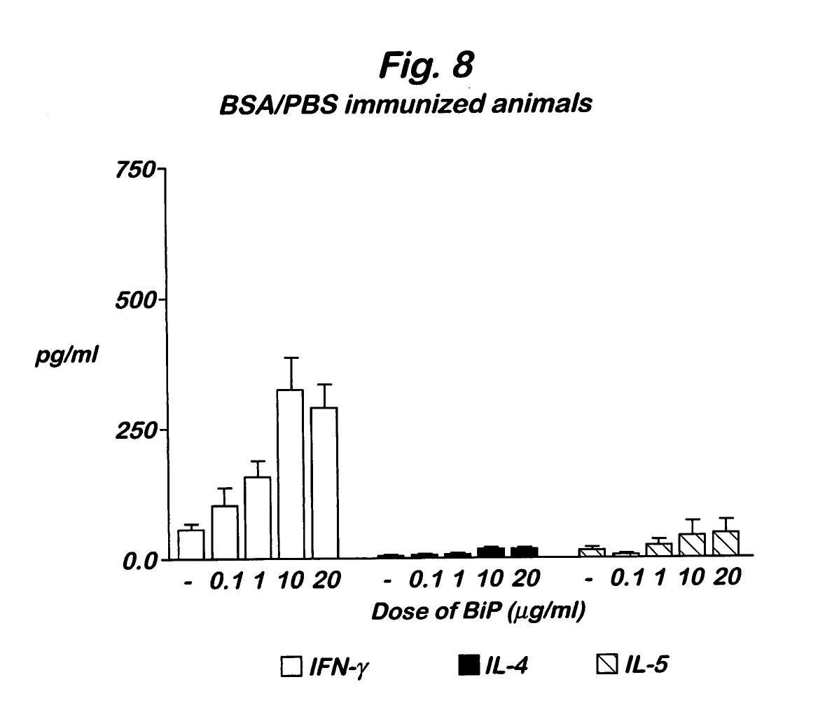

FIG. 8 shows BiP driven T cell cytokine production from control animals.

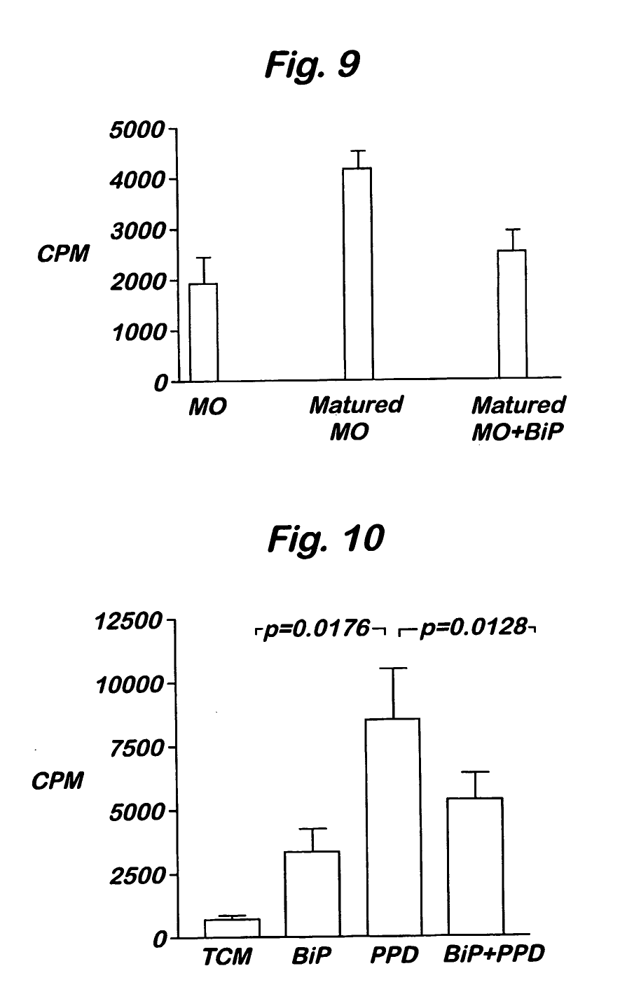

FIG. 9 shows the uptake of tritiated thymidine following an allogeneic reaction between peripheral blood monocytes (MO) either cultured for 5 days in tissue culture medium, or matured with granulocyte macrophage-colony stimulating factor (GM-CSF)+IL-4, or with GM-CSF+IL-4+BiP, prior to irradiation and culture with allogeneic peripheral blood mononuclear cells.

FIG. 10 shows the proliferation response of peripheral blood mononuclear cells measured by the uptake of tritiated thymidine in either unstimulated cultures (TCM) or cultures stimulated with BiP (20 μg/ml) or tuberculin PPD (10 μg/ml) or with BiP and PPD.

### EXAMPLES

Materials and Methods

Fluorescein Isothiocyanate Labelling of Proteins

BiP or human serum albumin (HSA) were prepared at a concentration of 2 mg/ml in carbonate buffer 0.1M pH9.6. A stock solution of fluorescein isothiocyanate (FITC) was prepared at 10 mg/ml in carbonate buffer 0.1M pH 9.6. 50 μg FITC/mg protein was added to the protein solution in a glass container covered in foil. The solution was placed on a circular mixer and incubated at room temperature for 2 hours. The FITC labelled protein was then placed in dialysis tubing (which had been boiled for 5 mins with each of three changes of fresh distilled water) and dialysed overnight in 5 litres of phosphate buffered saline (PBS) (0.15M NaCl, 4 mM NaH₂PO₄, 0.01M Na₂HPO₄ pH 7.2) followed by two further changes of 5 litres PBS. FITC labelled proteins were aliquoted and stored at 4° C.

Immunfluorescent Staining of Cells

PBMC were separated on Lymphoprep (Nycomed Amersham, Amersham, UK) by density centrifugation at 800 g and then washed three times in Hanks buffered saline solution (Life Technologies, Paisley UK). Fibroblast-like synoviocytes or other adherent cells grown in culture were gently scraped from the surface of the flask before immunofluorescenct staining. The cells were pelleted and resuspended in PBS containing 0.5% bovine serum albumin and 0.01% azide (PBS/BSA/az). Cells at 10⁵-10⁶/ml were used for the staining. 100 μl of cells were placed in a tube and 10 μl of ⅕ normal human serum added. The cells are incubated on ice for 10 minutes and then washed twice at 300 g in PBS/BSA/az at 4° C. The required amount of the FITC conjugated protein was then added to the cells in conjunction with any other protein directly conjugated to a different fluorochrome, such as phycoerythrin (PE), and the tube vortexed. The actual amount of protein added must be determined for each conjugation by a dose response curve. The cells were incubated on ice for 20 minutes and then washed twice at 300 g in PBS/BSA/az at 4° C.

BiP.FITC was used at 1/50 dilution and counter-stained with anti-CD20 (B cell marker), CD3, CD4, CD8 (T cell markers), CD56 (NK cell marker), or CD14 (monocyte marker) all directly PE conjugated and used at 5 μl/100 μl (Becton Dickinson, Oxford, UK). After the final wash the cells were fixed in PBS/BSA/az with 1% paraformaldehyde in 250 μl aliquots. The cells were then analysed on a FACScan using Cellquest software (Becton Dickinson, Oxford, UK).

Determination of Cytokine Production by PBMC Stimulated with BiP

Supernatants from cultures PBMC (10⁶1/ml) either unstimulated or stimulated by BiP (20 μg/ml) were harvested after 24 hours incubation. All cytokines were measured by commercial ELISA obtained from Pharmingen, Oxford, UK following 24 h stimulation.

Cloning Procedure for BiP Responsive Cells

Cloning of Specific T Cells:

Mononuclear cells were plated at 1×10⁶ cells ml⁻¹ in 2 ml culture wells in the presence of 20 μgml⁻¹ BiP (this concentration has been previously shown to be optimal for proliferation) in culture medium (RPMI 1640+10% human serum, L-glutamine and penicillin and streptomycin. Cells were cultured at 37° C. in 5% CO₂. After 7 days

Lymphocult-T (LC-T) was added to the cultures (40 μlml⁻¹) as a source of interleukin-2 (IL-2). After a further 7 days 1×10⁶ irradiated autologous feeder cells were added to each well with 40 μlml⁻¹ LC-T and 20 μgml⁻¹ BiP. This regime was continued for 3 rounds of feeder cells and then the cells plated at 10 cells per well into 96U plates with 1×10⁴ γ-irradiated allogeneic feeder cells (4000 Rads: ¹³⁷Cesium source) and 2 μgml⁻¹ Phytohaemagglutinin (PHA). After one week LC-T was added to the wells (40 μlml₋₁) and after a further week 1×10⁴ irradiated feeder cells, Lymphocult-T and PHA was added again. The cells were expanded into progressively bigger wells using this regime, until sufficient cell numbers were achieved for further study.

Proliferation Assays.

When sufficient cell numbers were achieved 1×10⁴ cloned cells were incubated for three days with 1×10⁵ irradiated autologous feeder cells in the presence or absence of BiP (20 μgml⁻¹) or PHA (2 μgml⁻¹).

The cells were incubated for the last 18 hours with ³H-thymidine (0.2 μCi) and then harvested. Proliferation was expressed as a stimulation index (SI): proliferation in the presence of stimulant/proliferation in the presence of medium alone.

Determination of Clonality:

Phenotypic analysis was carried out on responding clones using CD3, 4 and 8 (BD). Briefly, cells were washed in FACS buffer (phosphate buffered saline containing 1% bovine serum albumin and 0.05% sodium azide) and incubated with 4 μl of the appropriate antibodies. Three-colour analysis was performed using a FACScan flow cytometer and cell-quest software. T cell receptor usage was determined using a panel of both FITC conjugated and non-conjugated antibodies. Briefly, for conjugated antibodies 1×10⁴ cells were incubated with 4 μl of each of the FITC conjugated V beta specific antibodies. Clones were also stained with 4 μl of both anti-CD4 conjugated to FITC and CD8 conjugated to PE. Where non-conjugated antibodies were used cells were incubated for 40 mins on ice with the primary antibody, washed twice in FACS buffer, then incubated for a further 40 mins with a FITC conjugated goat antibody raised against mouse immunoglobulins.

Stained cells were run on a FACScan flow cytometer with a 488 nm laser and the results analysed using Cellquest and WinMDI analytical software.

Cytokine Determination.

Supernatants were removed from cultures 24 hours after the last round of stimulation. Supernatants from cultures containing only irradiated feeders, LC-T and PHA were used as controls. The amount of interleukin (IL) 4 and IL-1, and γ-interferon and tumour necrosis factor-α were determined by ELISA (Pharmingen, according to manufacturers instructions). Briefly, plates (Nunc Maxisorp) were coated with a cytokine specific capture antibody, blocked with 10% foetal calf serum (FCS: to stop non-specific binding) and the supernatants incubated on the plate overnight at 4° C. Bound cytokine was detected with a biotin conjugated detection antibody and visualised with streptavidin conjugated horseradish peroxidase and TMB.

Inhibition of Recall Antigen Responses by PBMC Preincubated with BiP

PBMC were cultured either alone or with BiP (20 μg/ml) for 0, 24, 48 or 72 hours prior to the addition of tuberculin purified protein derivative (PPD)(10 μg/ml) and the cells incubated for 6 days following the addition of PPD. Tritiated thymidine was added to the cultures for the final 6 hours. The cells were then harvested and the uptake of tritiated thymidine assayed using a dry matrix beta counter (Canbarra-Packard, Pangbourne, UK).

Cytokine Expression Array:

Monocytes (MO) were separated by negative selection using an immunomagnetic bead kit (Dynal, Wirral, UK) and placed in culture at 2.10⁶/ml for 24 hours either alone or stimulated with BiP(20 μg/ml) or with PMA (10 ng/ml)+IONO(250 ng/ml). The supernatants were harvested for ELISA and the cells were processed for extraction of total RNA and production of cDNA using oligo d(T) primers and reverse transcriptase. The expression array (R&D Systems, Oxford, UK) was used according to the manufacturer's instructions.

Mechanism of Action of BiP in Preventing AA and CIA

A Stimulating Release IL-10 from Human CD14+ Monocytes

The ability of BiP to prevent CIA and AA suggested to the inventors that it may have a generic ability to downregulate immune responses. In order to test this possibility, BiP was conjugated to fluorescein isothiyocyanate (FITC) and used in flow cytometry. As can be seen from FIG. 2, BiP binds to different populations of cells found in human peripheral blood but especially to human CD14+ monocytes.

When peripheral human blood mononuclear cells were cultured with BiP, cells were stimulated to release interleukin 10 (IL-10) as well as tumour necrosis factor (TNF)α (see FIG. 3). It is important to note that the amounts of secreted IL-10 are significantly greater than those for TNFα. Since IL-10 is anti-inflammatory and downmodulates immune responses while TNFα is pro-inflammatory, the administration of BiP will lead to the induction of an anti-inflammatory and downmodulatory immune environment thus explaining its ability to prevent the induction of CIA and AA.

B Stimulating Release from CD8+ T cells

CD8+ cells from peripheral blood of humans subjects may be stimulated by BiP to proliferate (FIG. 4). Indeed, the inventors have generated clones of such CD8+ T cells. These clones do not secrete the pro-inflammatory cytokine interferon (IFN)γ but do secrete IL-10 (FIG. 5). Thus these cells have the characteristics of Tcl regulatory CD8+ T cells and are able to downmodulate immune responses.

C Inhibition of Recall Antigen Responses by PBMC Pre-Treated with BiP

Pre-incubation of PBMC with BiP induced inhibition of the response to tuberculin purified protein derivative (PPD), a recall antigen. The inhibition increased with the length of time the cells had been exposed to BiP prior to the PPD challenge. (Inhibition of PPD responses: PBMC+BiP, Oh, 44.5±30%, range 0-67%; 24 h, 47.2±35.8%, range 0-87%; 48 h, 59.2±27%, range 30-90%; 72 h, 64±23.6%, range 33-90%)

D BiP Induces Monocytes to Activate a More Anti-Inflammatory Array of Genes Compared with Activation by Phorbol Myristic Acid (PMA) and Calcium Ionophore (IONO).

An expression array (R&D Systems, Oxford, UK) capable of screening 375 different genes was used to analyse the gene activation profile of resting monocytes or that following BiP or PMA+IONO activation. The preliminary results show that those genes activated by BiP were more anti-inflammatory than those activated following direct cell activation by PMA+IONO. Differences are shown in Table 1. To be noted should be the following facts: BiP specifically induced gene activation for monocyte migration inhibitory factor (MIF), soluble TNF receptor II and IL-10 anti-inflammatory mediators, upregulated tissue inhibitor of matrix metalloproteinases (TIMP) but did not induce matrix metalloproteinase (MMP) or monocyte chemoattractant protein (MCP)-1, potent pathogenic mediators, or the inflammatory cytokine, TNFα. In contrast, PMA+IONO activation of monocytes upregulated many inflammatory mediators, such as TNF α, MCP-1 and MMP-1, 9 and -10.

TABLE 1

| | Rest MO | BiP MO | BiP MO | PMA/IONO MO |
|---|---|---|---|---|
| Chemokines and | | | | |
| receptors | | | | |
| ENA-78 | Y** | Y | Y | Y |
| MIP-1alpha | | Y* | Y | Y |
| MIP-1beta | | Y* | Y | |
| GRO alpha | Y | Y | Y | Y* |
| GRO beta | Y | Y | Y | Y* |
| GRO gamma | Y | Y | Y | Y* |
| IL-8 | Y* | Y* | Y* | Y** |
| MCP-1 | | | | Y |
| MCP-3 | | | | Y |
| CCR1 | | | | Y |
| CXCR4 | | | Y | |
| LDGF | Y | Y | Y | |
| PARC | | Y | | Y |
| Orphan receptors | | | | |
| RDC-1 | | Y | Y | |
| ChemR23 | | Y | Y | Y |
| PARC | | Y | | Y |
| Proteinases or | | | | |
| related factor | | | | |
| Caspase | | Y | | Y |
| MMP-1 | | | | Y |
| MMP-9 | | | | Y |
| MMP-10 | | | | Y |
| TIMP-1 | Y | Y | Y | Y |
| Urokinase R | Y | Y* | Y* | Y |
| Caspase 1 | | Y | | Y |
| Integrins | | | | |
| Integrin beta 1 | Y | Y | Y | Y |
| Integrin beta-2 | Y | Y | Y | Y |
| Integrin beta-4 | Y | | Y | Y |
| TGF superfamily | | | | |
| Activin A | Y | Y | Y | Y* |
| TNF superfamily | | | | |
| LIGHT | Y | Y | Y | Y |
| Cytokines and | | | | |
| inhibitors | | | | |
| IL-1beta | Y** | Y** | Y** | Y** |
| IL-1Ra | Y | Y | Y | Y |
| IL-6 | Y | Y | Y | Y |
| IL-10 | | Y | | |
| TNF alpha | | | | Y |
| GM-CSF | | | | Y |
| MIF | | Y | Y | |
| Cytokine receptors | | | | |
| IFNgamma RI | Y | | | |
| IFNgamma RII | Y | | | Y |
| IL-7R alpha | Y | Y | Y | |
| TNFRII | | Y | | |
| CD14 | Y* | Y | Y | Y |

Expression array of the genes activated in resting monocytes (REST MO), or stimulated by BiP(BiP MO), or by PMA+IONO (PMA+IONO MO). Y indicated the gene was activated and mRNA was present, * indicated a high level of expression. Only the positive genes have been included in the table.

In Vitro Functional Studies with BiP

The functional consequences of the addition of BiP has been investigated in two important experimental systems:

1) The allogeneic reaction is the in vitro equivalent of transplantation

2) Tuberculin purified protein derivative (PPD) stimulation is a measure of the lymphocytes response to recall antigens (an antigen to which the subject has already been immunised).

FIG. 9 shows that allogeneic peripheral blood lymphocytes react to the resting monocytes with a low response. When the monocytes are matured into dendritic cells, using the well established technique of adding granulocyte macrophage colony stimulating factor (GM-CSF) and interleukin-4 (IL-4) for 5 days, the response by the allogeneic PBMC is greatly increased. When BiP is added with GM-CSF+IL-4 at the start of monocyte maturation the allogeneic response by PBMC is decreased to that of the background control.

These findings support the concept that BiP given before organ, tissue, cell, gene or protein administration will prevent immunisation. In terms of transplantation this should lead to acceptance of the graft.

FIG. 10 shows that the PPD response by PBMC is significantly reduced to background levels when BiP is added to the PPD cultures.

These findings support the concept that the administration of BIP will suppress ongoing inflammatory and autoimmune diseases.

In Vivo Experiments

In developing BiP as a therapeutic drug two important properties need to be ascertained: first, the nature of the cytokines released after parenteral administration of BiP and, second, the optimum dose of BiP needed to achieve the maximum release of cytokines

1 BiP as an Immunomodulator

Method: Male DBA-1 mice (8-12 weeks old) were immunised subcutaneously (s.c.) with 200 μg of BiP in phosphate buffered saline (PBS). PBS alone or bovine serum albumin (BSA) were administered as controls. 14 days later, spleens and lymph nodes were removed and T cell cultures set up and stimulated with varying concentrations of BiP at 0.1, 1, 10, 20 μg/ml. After 4 days of culture, the pro-inflammatory cytokine interferon (IFN)-γ and the anti-inflammatory cytokines interleukin (IL)4, IL-5 and IL-10 were assayed. T cell proliferation was assessed by ³H-thymidine incorporation.

Results: T cells derived from animals immunised with BiP proliferated in a dose dependent manner on in vitro stimulation (FIG. 6, BiP). Control animals exhibited modest proliferation to in vitro stimulation with BiP (FIG. 6, PBS). The cytokine profiles also demonstrated a dose dependent increase in IL-4 and IL-5 production by T cells derived from all BiP immunised mice (levels ˜500 pg/ml and ˜700 pg/ml respectively) (FIG. 7) compared to the control animals (<100 pg/ml) (FIG. 8). However, only modest levels of IFN-γ were produced by T cells derived from BiP-only immunised mice.

Conclusion: BiP is surprisingly immunogenic and immunising mice with BiP results in a raised anti-inflammatory cytokine profile with a switch towards a TH2 profile.

2 Optimising the BiP Immunisation Dose

Methods: Groups of mice were immunised with 4 different doses of BiP (either 50, 200, 500 μg or 1 mg), and 14 days later, spleen and lymph nodes were removed and the cells assayed for proliferation and cytokine secretion after 4 days in culture with BiP.

Results: It was evident from these experiments that the optimal immunising dose of BiP was 500 μg as T cells derived from mice re-stimulated in vitro with 10 μg/ml BiP proliferated most vigorously (600,000 CPM) whereas T cell proliferation from all other immunisation groups were below 40,000 CPM. Cytokines studies were in agreement showing levels of IL-4 reaching ˜800 pg/ml in mice immunised with 500 μg (below 500 pg/ml in all other groups). Similarly IL-5 levels reached ˜600 pg/ml in the 500 μg group and did not reach over 400 pg/ml in any of the other groups. T cells from naïve mice proliferated very slightly to BiP stimulation, produced very low quantities of Th2 cytokines and ˜1000 pg/ml of IFN-γ.

Conclusion: The optimum proliferation and cytokine secretion upon in vitro BiP stimulation was observed when mice were immunised with 500 μg of BiP.

1. The use of BiP or a functional fragment or homolog thereof, or a nucleic acid molecule encoding BiP or a functional fragment or homolog thereof, in the manufacture of a medicament for the treatment or prevention of an unwanted immune response.

2. The use according to claim 1, wherein the unwanted immune response is associated with an immune-mediated disease.

3. The use according to claim 2, wherein the immune-mediated disease is an auto-immune disease.

4. The use according to claim 2, wherein the immune-mediated disease is type 1 diabetes, thyroiditis or multiple sclerosis, systemic lupus erythematosus, Crohn's disease and all forms of viral and autoimmune hepatitis.

5. The use according to any one of claims 2 to 4, wherein the medicament additionally comprises an agent for enhancing the treatment or prevention of the immune-mediated disease.

6. The use according to any one of claims 2 to 5, wherein the medicament is administered to an individual suffering from or susceptible to developing an immune-mediated disease.

7. The use according to claim 6, wherein the medicament is administered to the individual prior to the development of the immune-mediated disease or as soon as the immune-mediated disease has be diagnosed.

8. The use according to claim 1, wherein the unwanted immune response is associated the rejection of a transplanted organ, a tissue or cells.

9. The use according to claim 8, wherein the medicament additionally comprises an agent for enhancing the treatment or prevention of transplant rejection.

10. The use according to claim 9, wherein the agent is a suppressor of the immune system.

11. The use according to any one of claims 8 to 10, wherein the medicament is administered to an individual before or at substantially the same time as the transplantation of an organ, tissue or cells.

12. The use according to claim 1, wherein the unwanted immune response is the immune response to a biologic.

13. The use according to claim 12, wherein the biologic is a therapeutic protein, glycoprotein or carbohydrate.

14. The use according to claim 12 or claim 13, wherein the medicament additionally comprises an agent for enhancing the treatment or prevention of the immune response to the biologic.

15. The use according to any one of claims 12 to 14, wherein the medicament is administered to an individual before or at substantially the same time as the biologic.

16. The use of BiP or a functional fragment or homolog thereof, or a nucleic acid molecule encoding BiP or a functional fragment or homolog thereof, for stimulating the release of IL-0 from cells capable of releasing IL-10.

17. The use according to claim 16, wherein BiP or a functional fragment or homolog thereof, or a nucleic acid molecule encoding BiP or a functional fragment or homolog thereof, additionally stimulates gene expression of at least one of monocyte migration inhibitory factor (MIP), soluble TNF receptor II, IL-10 anti-inflammatory mediators and tissue inhibitor of matrix metalloproteinases (TIMP).

18. The use according to claim 17, wherein BiP or a functional fragment or homolog thereof, or a nucleic acid molecule encoding BiP or a functional fragment or homolog thereof, does not stimulate gene expression of matrix metalloproteinases (MMPs), monocyte chemoattractant protein (MCP-1) or TNFα.

19. A pharmaceutical preparation comprising BiP or a functional fragment or homolog thereof, or a nucleic acid molecule encoding BiP or a functional fragment or homolog thereof, in combination with a pharmaceutically acceptable carrier for use in the treatment or prevention of an unwanted immune response.

20. The pharmaceutical preparation according to claim 19, wherein the unwanted immune response is associated with an immune-mediated disease; associated with the rejection of a transplanted organ, tissue or cell; or is the immune response to a biologic.

21. The pharmaceutical composition according to claim 19 or claim 20, which additionally comprises an agent for enhancing the treatment of prevention of the unwanted immune response.

22. A method of treating or preventing an unwanted immune response comprising administrating to an individual in need of such treatment an effective dose of the pharmaceutical composition according to any one of claims 19 to 21.

23. The use of BiP or a peptide fragment thereof (including a synthetic peptide) for stimulating the release of Interleukin 10 (IL-10).

24. The use according to claim 23, for the preparation of a medicament for the treatment of auto-immune disease.

25. The use according to claim 24, for the preparation of a medicament for the prevention of rejection of transplanted organs, tissues, or cell.

26. The use according to claim 23, for the preparation of a medicament for the prevention of an immune response to biologic therapeutic substances.

27. The use according to claim 23, for the preparation of a medicament for the treatment of immune-mediated disease, including type 1 diabetes, thyroiditis, and multiple sclerosis, systemic lupus erythematosus, Crohn's disease and all forms of viral and autoimmune hepatitis.

28. Pharmaceutical preparations comprising BiP or a fragment thereof adapted for treatment of the diseases or situations referred to in any of claims 23 to 27.

29. A method of treatment of the diseases or situations referred to in any of claims 23 to 27, which comprises administering to a patient in need thereof. BiP or a fragment thereof.
